(19) United States
(12) Patent Application Publication
(10) Pub. No.: US 2013/0204332 A1
(43) Pub. Date: Aug. 8, 2013

(54) BODY COOL HARNESS

(71) Applicant: Michael Amalfi, Pittsford, NY (US)

(72) Inventor: Michael Amalfi, Pittsford, NY (US)

(73) Assignee: Body Cool LLC, Rochester, NY (US)

(21) Appl. No.: 13/876,930

(22) PCT Filed: Sep. 28, 2011
(86) PCT No.: PCT/US2011/053559

§ 371 Date: Apr. 15, 2013

Related U.S. Application Data
(60) Provisional application No. 61/387,496, filed on Sep. 29, 2010.

(51) Int. Cl. A61F 7/10

(52) U.S. Cl. CPC A61F 7/10
USPC 607/112

(57) ABSTRACT

A body cool harness is disclosed. The body cool harness may have a body portion for overlaying the back region of a person, a first wrap-around portion, and at least one cooling device.

This application is a submission under 35 U.S.C. 371 and claims priority to PCT/US2011/053559 filed Sep. 28, 2011 and to U.S. Provisional Application Ser. No. 61/387,496 filed Sep. 29, 2010.

### FIELD OF THE INVENTION

This invention relates to the field of emergency medical devices, including the use of cooling devices embedded within medical equipment.

### BACKGROUND OF THE INVENTION

Great interest is currently being generated in the field of spinal cord injury, heart attack, stroke, and other traumatic injury treatment by the use of cooling to control swelling and prevent further injury. The efficacy of this treatment increases as the time between initial injury and application of the treatment decreases. Therefore, it is desirable to have equipment for use at the scene of the injury which will enable such treatment.

### BRIEF SUMMARY OF THE INVENTION

A medical device according to the invention includes one or more cooling devices. The medical device may be constructed as a brace to fit around a patient's body. Such medical devices may be used to prevent swelling in an injured person. The cooling devices may facilitate cooling of the back and arteries to reduce swelling of the brain. Reduction of swelling of the brain helps to decrease both short and long term damage to the brain of a patient.

In an embodiment of the invention, the medical device may be in the form of a brace that wraps around a patient's body and contains a plurality of cooling devices. One such cooling device has an impermeable bag and a selectively breakable barrier, which effectively divides the cooling device into two storage areas. One of the storage areas may be filled with water, and the other storage area may be filled with a chemical that reacts with water to produce an endothermic reaction.

Another cooling device has an impermeable outer bag and a selectively breakable inner bag. The outer bag may include water and the inner bag may include a chemical which will react with the water to produce an endothermic reaction. Alternatively, the inner bag may include water and the outer bag may include the chemical. The chemical (“reactant”), such as ammonium nitrate, reacts with water to produce an endothermic reaction.

In use, the selectively breakable barrier or bag, as the case may be, is broken in order to allow the reactant to react with the water thereby causing an endothermic reaction. This reaction will cool the water contained within the impermeable bag, the bag itself, and any objects in thermal communication with the impermeable bag.

The present invention may be directed to a medical device that can be affixed to a human body. The medical device may have a plurality of cooling devices to cool particular areas of a patient, such as a patient's back, lower neck, armpits, and groin. The body cool harness may be adapted to allow the initiation of the reaction before or after the medical device is affixed to an injured person. The medical device may be foldable and/or adapted to fit inside a carrying suitcase. Multiple cooling assemblies may be used in this device.

The cooling devices may be attached or disposed within a fabric that follows the general contours of a patient's body. Straps may be used to attach the body cool harness to a patient. For example, straps may wrap-around a patient's upper torso, waist, and/or legs. Such straps may use hook-and-loop fasteners to secure the medical device to a patient. The medical device may also include multiple fabrics and multiple layers of fabric to facilitate an adjustable and transportable cooling medical device to treat a patient suffering from physical trauma.

### BRIEF DESCRIPTION OF THE DRAWINGS

The invention will be described further by way of non-limiting examples, with reference to the attached drawings and diagrams in which:

FIG. 1 shows a plan view of the body cool harness;

FIG. 2 shows an expanded view of the leg portion of the body cool harness;

FIG. 3 shows a plan view of the body cool harness with a fabric breakdown;

FIG. 4 is a side view of the body cool harness;

FIG. 5 is a side view of the body cool harness which identifies fabrics that could be used;

FIG. 6 is a layer view of the body cool harness which identifies fabrics that could be used;

FIG. 7 is a bottom view of the body cool harness;

FIGS. 8A and 8B depicts the body cool harness as it might be positioned on a user; and

FIGS. 9A and 9B depict a carrying case for the body cool harness (closed position in 9A and open position in 9B).

### DETAILED DESCRIPTION OF THE INVENTION

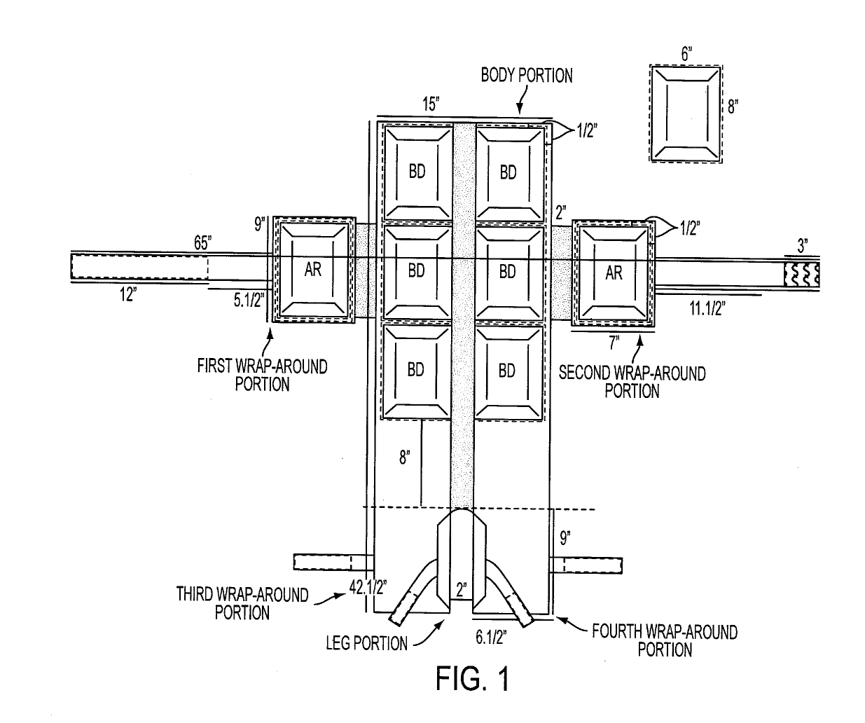

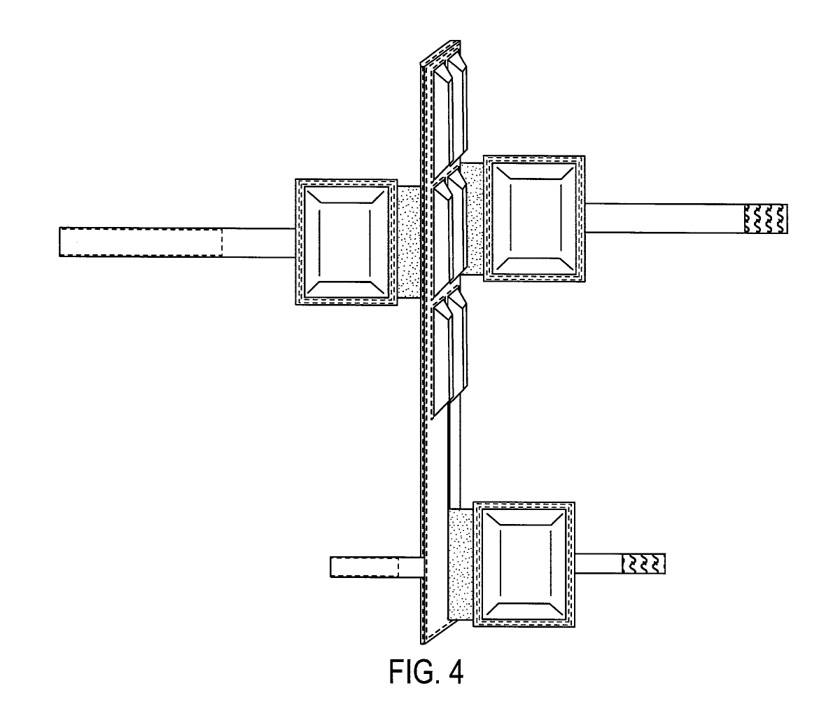

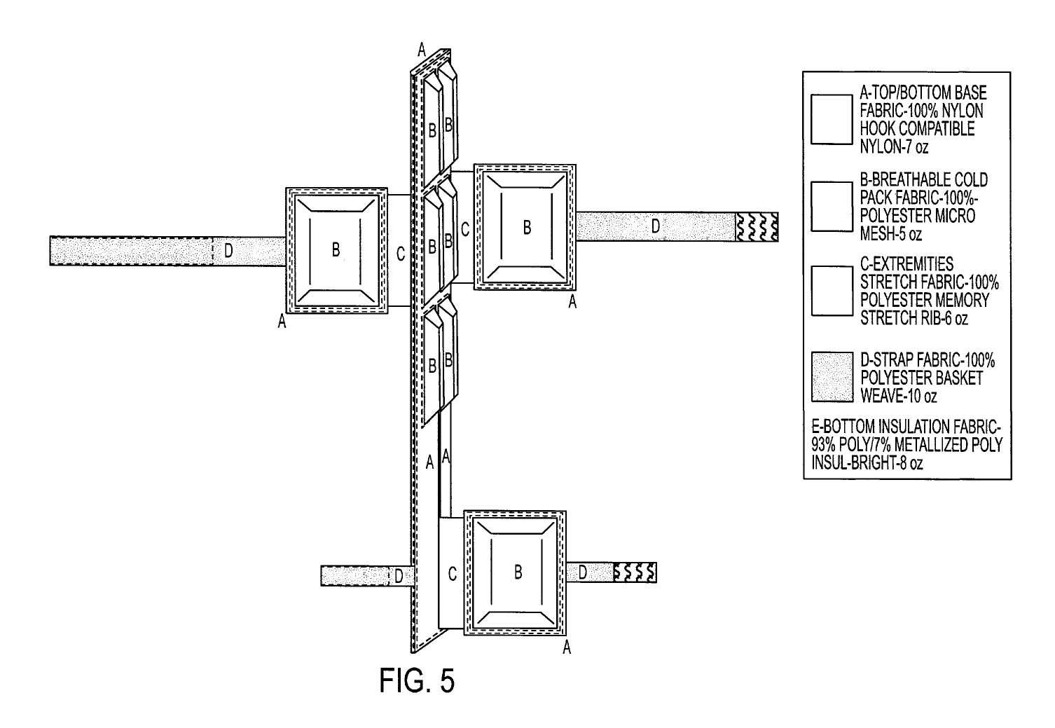

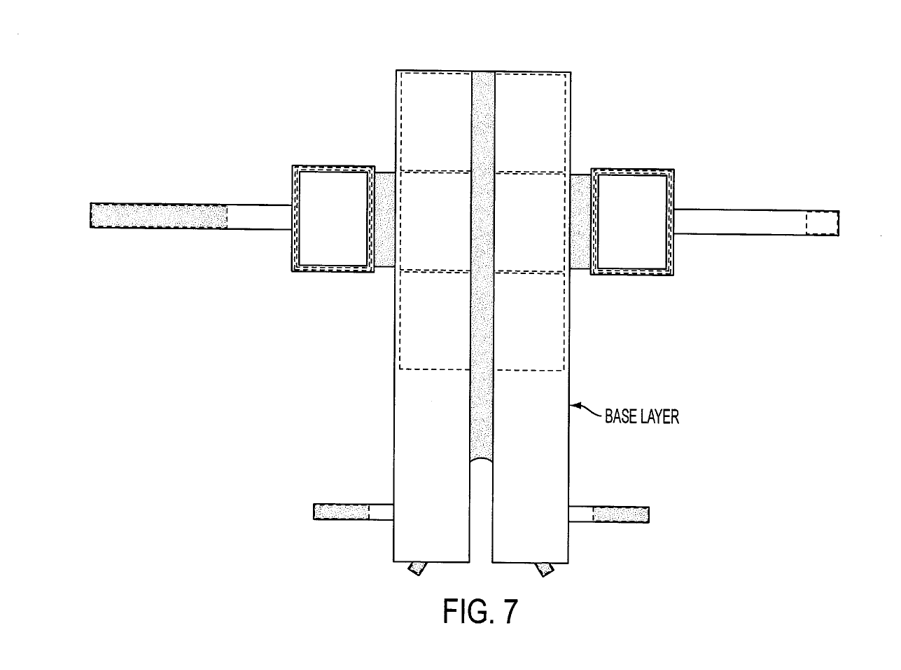

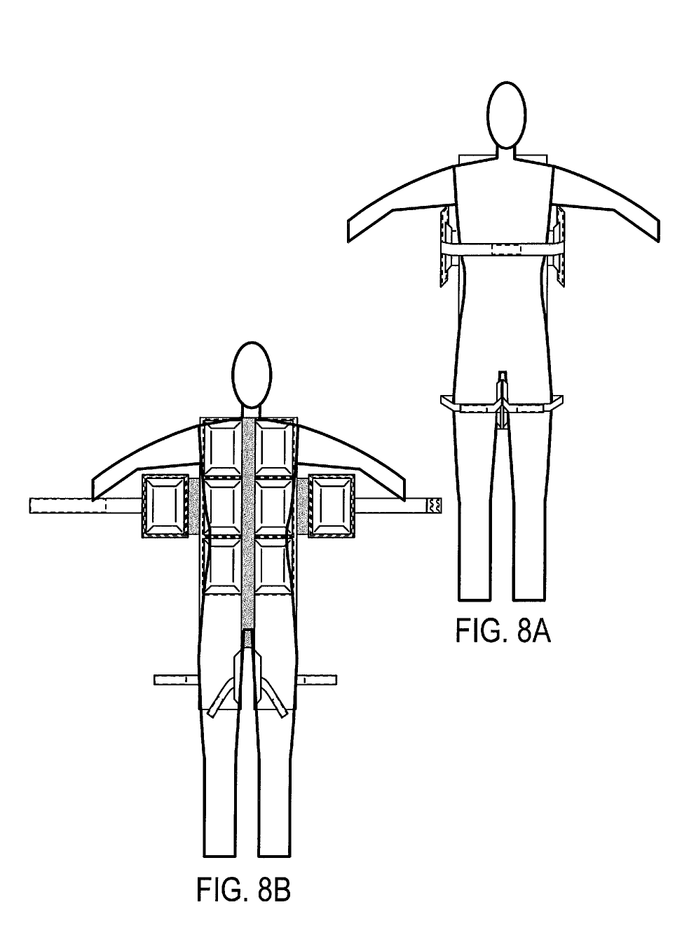

FIGS. 1-8 show embodiments of a body cool harness that is in keeping with the invention. The body cool harness may be arranged as part of a wrapable brace. In FIG. 1 there is shown a body cool harness having a body-portion, a first wrap-around portion, a second wrap-around portion, a third wrap-around portion, and a fourth wrap-around portion. The wrap-around portions may be used to cool a person's armpits and/or groin. The first wrap-around portion may be placed in a first armpit and the second wrap-around portion may be placed in a second armpit. The third wrap-around portion and the fourth wrap-around portion may be placed on each side of a person's groin.

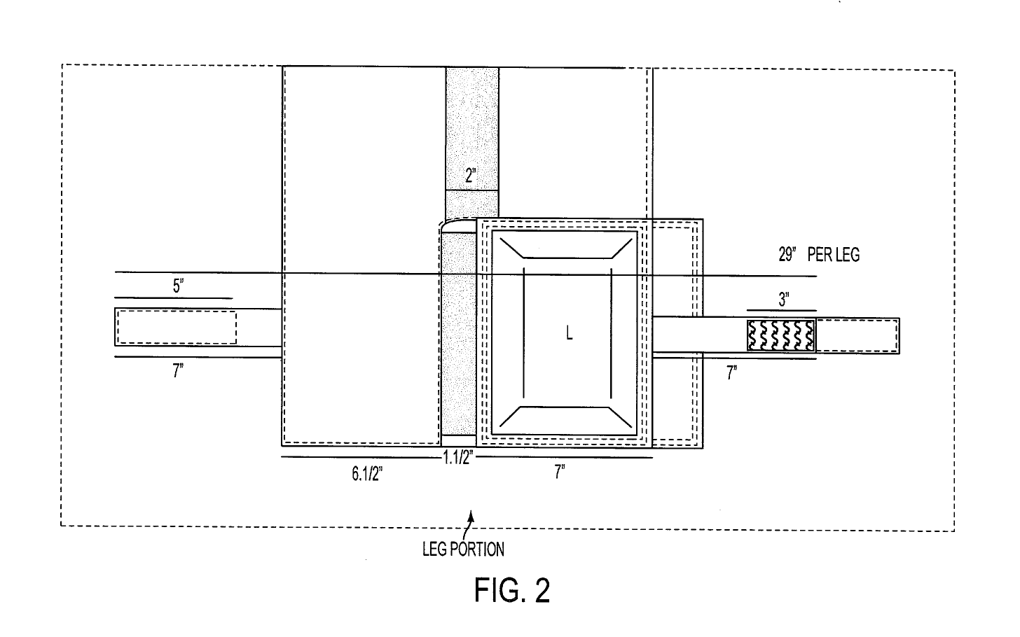

In one or more portions of the body cool harness there may be a cooling device. For example, the embodiment of FIG. 1 has six cooling devices that correspond to the body portion and one cooling device for each wrap-around portion. In FIG. 1, the cooling devices that correspond to the body portion are labeled “BD” and the cooling devices in the first wrap-around portion and the second wrap-around portion are labeled “AR.” As may be seen in FIG. 2, a cooling device that corresponds to the third portion is labeled “L.” The remainder of the drawings are not labeled to indicate the cooling devices that correspond to each portion.

The cooling device may include an impermeable bag having a selectively breakable barrier therein. The barrier divides the bag into a first package having water therein, and a second package having a reactant, such as ammonium nitrate. Prior to breaking the barrier is broken, the contents of the first package are allowed to mix with the contents of the second package. By mixing the contents of the packages, an endothermic reaction is made to occur by the reaction of the contents. This reaction will cool the contents of the cooling device and any objects that are in thermal communication with the cooling device, including a person's body.

The barrier may be broken before the body cool harness is placed around a person's body. Alternatively, the barrier may be broken after a person's body is placed on the body cool harness, or at the same time a person's body is placed on the body cool harness. One method of breaking the barrier, in order to allow the reactant to mix with the water, is to apply pressure to the barrier. Pressure may be applied to the barrier by placement of the body on the cooling device or by force applied to the barrier by a user, such as a doctor, nurse, emergency medical technician, or other medical provider.

The body portion may be shaped to form to a person's back and legs. It may house a plurality of cooling devices within multiple layers of fabrics. Alternatively, the cooling devices may be attached externally to the body portion. The body portion may include a stretchable portion to facilitate adjustment to a person's body and/or allow for compression against a person's body. Extending from the body portion may be two leg portions where wraparound portions meet to cool a person's groin area, thereby cooling the blood traveling in the femoral vessels. Wrap-around portions may also extend from the body portion at a location that will allow these wrap-around portions to cool a person's arms or armpits, thereby cooling the blood traveling in the axillary vessels.

The wrap-around portions may be made from a flexible material so that they can be laid over a person who is attached to the body cool harness. In this manner, the person's torso, arms, arm pits, and/or groin may be cooled. The wrap-around portions may be oriented and positioned to wrap-around a person's shoulder-area, thereby allowing a cooling device to be placed in a person's arm pit. Alternatively, the wrap-around portions may be oriented and positioned to wrap against the arm, thereby allowing a cooling device to be placed in a person's armpit. The wrap-around portions may be oriented and positioned to wrap around the leg, thereby allowing a cooling device to be placed adjacent to a person's groin. Wrap-around portions may be located to cool major arteries such as the axillary artery and the femoral artery. In doing so, the person may be cooled more rapidly than otherwise and the femoral artery. In doing so, the person may be cooled more rapidly than otherwise would be the case.

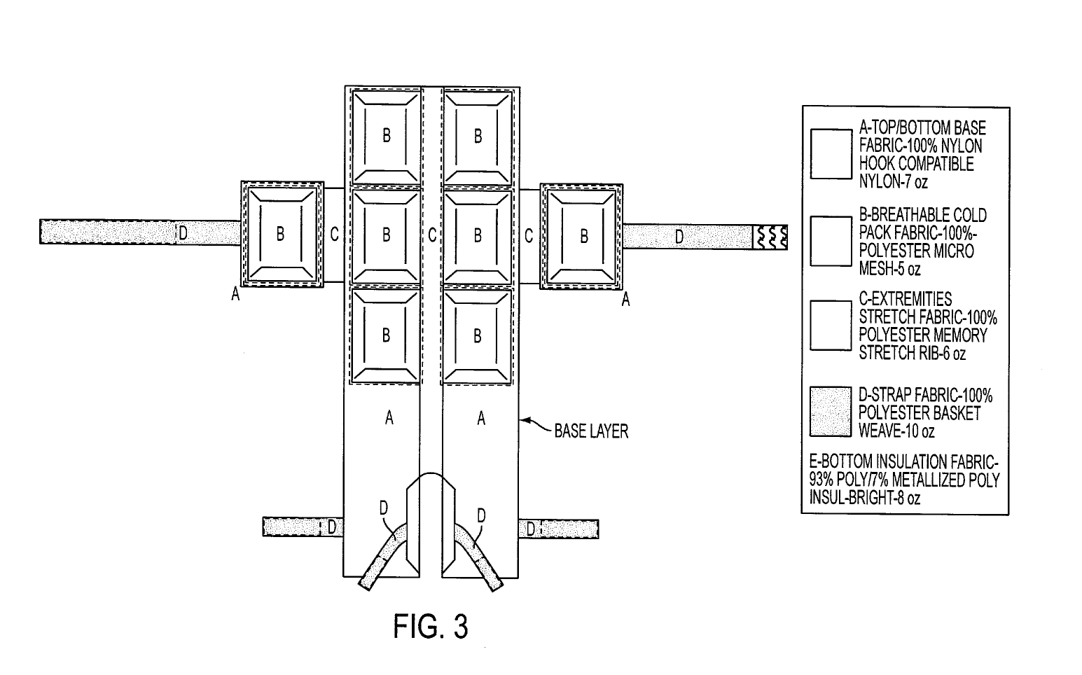

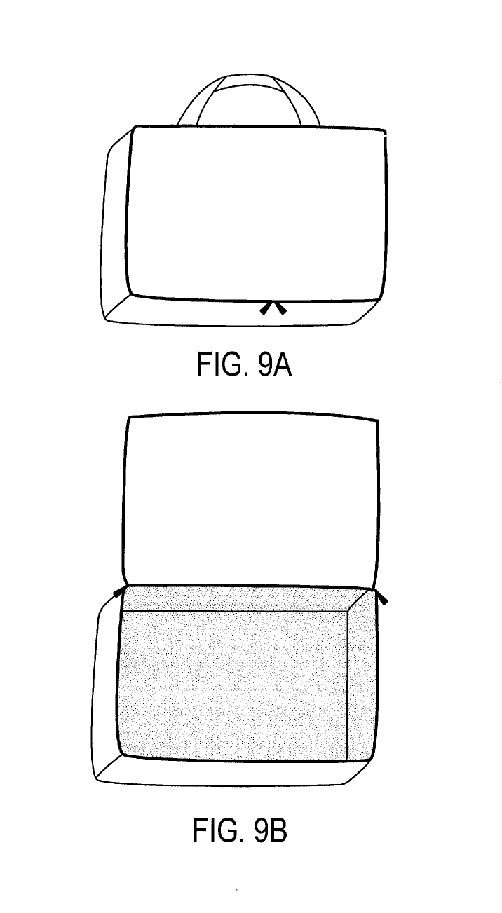

FIGS. 3, 5, and 6 identify fabrics which may be used in the body cool harness. A variety of fabrics and combinations of fabrics may be used, and these are merely examples. A base layer may form the foundation of the body cool harness. The base layer may be made from a durable fabric, such as polyester; however, alternative fabrics may be used. An insulating layer may be attached or sandwiched between base layers. The insulating layer may be made from an insulating fabric such as a polyester/metallized polyester blend; however, alternative fabrics may be used. The body cool harness is preferably flexible, so that it may be folded, and stored in a carrying case (FIGS. 9A and 9B).

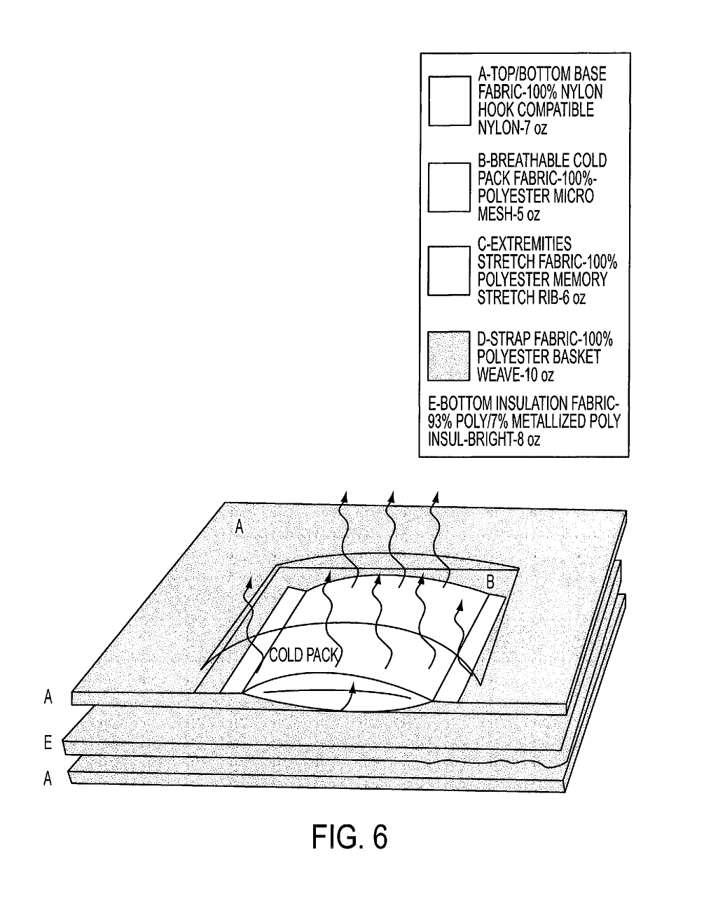

FIG. 6 shows that the base layer may have cut-out portions for housing cooling devices. These cut-out portions may be covered by a breathable layer to facilitate cooling. An insulating layer may be placed behind a cooling pack. An example of a suitable breathable layer material is micro-mesh polyester; however, alternative breathable and/or stretchable fabrics may be used. A stretchable layer may be made from stretch or memory stretch fabric. The stretchable layer may be used to connect base layers. A plurality of stretchable layers may increase adjustability of the body cool harness and facilitate compression against a person.

Straps may be used to allow for the cooling device to be affixed to a user. Straps may wrap-around a patient's upper torso, waist, and/or legs. Such straps may use hook-and-loop fasteners to secure the medical device to a patient; however, other types of fasteners may be used to secure the straps.

Although embodiments of the invention have been described herein, the invention is not limited to such embodiments. The claims which follow are directed to the invention, and are intended to further describe the invention, but are not intended to limit the scope of the invention.

What is claimed is:

1. A body cool harness having a body portion for overlaying the back region of a person, a first wrap-around portion, a second wrap-around portion, and at least one cooling device, wherein the first wrap-around portion and the second wrap-around portion fasten to one another with a strap.

2. The body cool harness of claim 1, further comprising a third wrap-around potion, and a fourth wrap-around portion, wherein the third wrap-around portion and the fourth wrap-around portion are configured to fasten around the legs of a person for overlaying the groin region of a person.

3. The body cool harness of claim 2, wherein the first, second, third, and fourth wrap around portions each include a cooling device.

4. The body cool harness of claim 3, wherein the body portion comprises an insulating fabric, a breathable fabric, a base fabric, and a stretchable fabric.

5. The body cool harness of claim 4, wherein the base fabric includes at least one cut out region for housing the cooling device.

6. The body cool harness of claim 5, wherein the cooling device is disposed between the insulating fabric and the breathable fabric.

7. The body cool harness of claim 6 wherein the stretchable fabric is disposed between base fabric.

8. The body cool harness of claim 7, wherein the cooling device has an impermeable bag having a selectively breakable barrier therein, thereby dividing the bag into a first package and a second package.

9. The body cool harness of claim 8, wherein the first package contains water.

10. The body cool harness of claim 9, wherein the second package contains a reactant, such as ammonium nitrate.

11. The body cool harness of claim 1, wherein the cooling device has a selectively breakable bag inside an impermeable bag.
